(19) United States
(12) Patent Application Publication
(10) Pub. No.: US 2024/0058386 A1
(43) Pub. Date: Feb. 22, 2024

(54) METHOD FOR IMPROVING JOINT HEALTH BY ADMINISTERING BOVINE MILK-DERIVED EXOSOMES

(71) Applicant: ABBOTT LABORATORIES, Abbott Park, IL (US)

(72) Inventors: José María LÓPEZ PEDROSA, Cenes de la Vega (ES); Ricardo RUEDA CABRERA, Granada (ES); Jorge GARCÍA MARTÍNEZ, Armilla (ES)

(21) Appl. No.: 18/259,874

(22) PCT Filed: Dec. 20, 2021
(86) PCT No.: PCT/US2021/064374

§ 371 Date: Jun. 29, 2023

(30) Foreign Application Priority Data
Dec. 31, 2020 (EP) 20383181.3

(51) Int. Cl. A61K 35/20; A61P 19/02

(52) U.S. Cl. CPC A61K 35/20; A61P 19/02

(57) ABSTRACT

A method of improving joint health in a subject in need thereof comprises administering an exosome-enriched product comprising intact bovine milk-derived exosomes to the subject in need thereof.

### FIELD OF THE INVENTION

The present invention relates to a method of improving joint health in a subject in need thereof by administering an exosome-enriched product comprising intact bovine milk-derived exosomes to the subject in need thereof.

### BACKGROUND OF THE INVENTION

Cartilage is a porous tissue composed largely of water (65-85% of total weight) and chondrocytes embedded in a complex extracellular matrix. Cartilaginous tissues are present throughout the body and are classified as elastic, fibrocartilaginous and hyaline based on their chemical composition. Among these, hyaline is the most abundant type of cartilage, being associated with the skeletal system. For instance, hyaline cartilage forms the growth plate but also covers the sliding surfaces at the end of bones in synovial joints, such as knees and hips. When hyaline cartilage is on the articular surfaces of bones, it is called articular cartilage. Together with the synovial fluid, articular cartilage is responsible for the lubrication of the joints and provides the properties needed to resist compressive loads enabling smooth articulation.

Articular cartilage is a connective tissue composed of chondrocytes. Chondrocytes are highly specialized cells that assist with the development, repair and maintenance of the extracellular matrix, which comprises type II collagen and proteoglycans. Type II collagen is the primary structural backbone of the matrix, with many molecules of the large proteoglycan aggrecan interacting with the collagen fibril network and hyaluronic acid through proteins known as link proteins.

Although cartilage is aneural and avascular, it is a dynamic tissue which is subjected to anabolic processes (chondroformation) and catabolic processes (chondroresorption). In healthy individuals, chondrocytes maintain a dynamic equilibrium between the biosynthetic and the catabolic processes. Aging, trauma and articular diseases, such as osteoarthritis, have been shown to cause a disturbance in this equilibrium, favoring catabolic processes over anabolic processes. This imbalance leads to a net decrease of extracellular matrix and results swelling, inflammation, pain, stiffness, joint degeneration and reduced mobility.

One of the most common diseases affecting articular cartilage is osteoarthritis, a degenerative condition that induces pain and impairs quality of life. Unfortunately, osteoarthritis affects adults as young as 20 to 30 years of age, and most adults over 60 years of age have osteoarthritis, although the severity varies. In the United States, the Osteoarthritis Action Alliance (OAAA), Arthritis Foundation (AF), and Centers for Disease Control and Prevention (CDC) have developed A National Public Health Agenda for Osteoarthritis: 2020 Update to address the high prevalence of osteoarthritis, its rising health impact and growing economic consequences. By 2040, the number of adults with arthritis is projected to increase to 78.4 million, most of whom will have osteoarthritis.

In degenerative joint diseases, such as osteoarthritis, there is a progressive loss of articular cartilage components. This phenomenon leads to the destruction of the connective tissue that ultimately disrupts joint function while provoking pain and impairing quality of life. Osteoarthritis has been shown to be hallmarked by long-lasting inflammatory changes that inhibit the synthesis of proteoglycans and collagen and enhance their degradation, thus disrupting the normal homeostasis of cartilage. The loss of proteoglycans from cartilage is a significant contributing factor to joint dysfunction, since proteoglycans hold moisture in the cartilage matrix and provide the osmotic properties needed to resist compressive loads and help structurally protect the cartilage from deterioration.

As osteoarthritis progresses, more proteoglycans are lost as a result of an impaired anabolic response of the chondrocytes. However, this decrease in chondrocyte anabolic function is not limited to pathological conditions. In fact, it has been reported that chondrocytes embedded within the cartilage matrix have minimal proliferative capacity and their ability to synthesize proteoglycans declines with age, thus contributing to diminished articular tissue integrity in the elderly.

Another condition, which is particularly common in the athletic population and has been observed with increasing frequency, is articular cartilage lesions of the knee. As mentioned above, articular cartilage is responsible for the lubrication of the joints and provides the properties needed to resist compressive loads enabling smooth articulation. Lesions in the articular cartilage of the knee result in friction in the joint, which in turn results in pain. Articular cartilage lesions can thus lead to progressive pain and functional limitation over time. If left untreated, isolated cartilage lesions can lead to progressive chondropenia or global cartilage loss over time. Nutraceuticals and chondro-protective agents are currently being investigated as tools to slow the development of chondropenia.

Currently, there are several approaches for restoration of damaged articular cartilage, including physical therapy, pharmacological treatment and surgical interventions. The goal of any reconstructive or regenerative procedure is to relieve the symptoms of the cartilage defect and restore joint function. However, there is currently a need for strategies to stimulate cartilage repair and recovery through stimulation of anabolic processes.

Increasing proteoglycan synthesis may represent an efficient way to recover/repair articular cartilage in conditions that are linked to an abnormal homeostasis of cartilage extracellular matrix, including, but not limited to, osteoarthritis, cartilage damage after trauma, articular cartilage lesions, and diminished cartilage integrity caused by normal aging. It is thus desirable to find new treatments that may help stimulate proteoglycan synthesis and thereby improve joint health, particularly in those subjects suffering from one or more of the aforementioned conditions.

### SUMMARY OF THE INVENTION

It is therefore an object of the invention to provide a method which improves joint health in a subject in need thereof.

The present invention is directed to a method of improving joint health in a subject in need thereof comprising administering an exosome-enriched product comprising intact bovine milk-derived exosomes to the subject in need thereof.

The methods of the invention are advantageous in providing a convenient manner to stimulate the anabolic activity of chondrocytes. By increasing chondrocyte proteoglycan synthesis, the methods of the invention represent a novel way to enhance cartilage repair and recovery in conditions such as osteoarthritis, cartilage damage after trauma, or diminished cartilage integrity cause by normal aging. The methods are also useful in the treatment of such conditions. These and additional advantages of the inventive methods will be more fully apparent in view of the detailed description.

### BRIEF DESCRIPTION OF THE DRAWINGS

The drawings are illustrative of certain embodiments of the invention and exemplary in nature and is not intended to limit the invention defined by the claims, wherein:

FIG. 1 illustrates the effect of bovine-milk derived exosomes on proteoglycan formation in human chondrocytes incubated with an exosome-enriched product containing intact bovine milk-derived exosomes, as described in Example 2.

FIG. 2. illustrates a flow diagram of a membrane filtration process coupled to spray-drying or freeze-drying to produce a lactose-free exosome-enriched product from cheese whey, as described in Example 1.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain embodiments of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

The term “exosome-enriched product comprising bovine milk-derived exosomes” as used herein, unless otherwise specified, refers to a product in which exosomes have been substantially separated from other bovine milk components such as lipids, cells, and debris, and are concentrated in an amount higher than that found in bovine milk. The exosomes are small, extracellular vesicles and account for a minor percentage of milk's total solids content. In specific embodiments, the exosome-enriched product is provided in a liquid form or a powdered form and also contains co-isolated milk solids.

The term “improving joint health” as used herein refers to reducing joint inflammation, regenerating cartilage tissue, strengthening cartilage tissue, and/or improving joint lubrication.

The term “intact exosomes” as used herein refers to exosomes in which the vesicle membrane is not ruptured and/or otherwise degraded and the endogenous cargo, i.e., the bioactive agents, therapeutics (e.g. miRNA), and/or other biomolecules which are inherently present in a bovine milk-derived exosome, are retained therein in active form.

The term “powdered exosomes” as used herein, unless otherwise specified, refers to a dry powder that contains exosomes which have been isolated from bovine milk. The isolated exosomes are dried to form a dry powder. As the isolated fluid containing the exosomes also contains co-isolated milk solids as described above, the powdered exosomes also contain such other milk solids in the resulting powder.

As indicated above, the present invention is directed to a method of improving joint health in a subject in need thereof. The method comprises administering an exosome-enriched product comprising intact bovine milk-derived exosomes to the subject in need thereof. Without wishing to be bound by any particular theory, the method of the present invention increases chondrocyte proteoglycan synthesis and thereby enhances cartilage repair and recovery via the administration of intact bovine milk-derived exosomes to the subject in need thereof. As indicated above, the methods of the invention are therefore useful in the treatment of conditions that are linked to an abnormal homeostasis of cartilage extracellular matrix.

The enriched product of intact bovine milk-derived exosomes is typically obtained from a whey fraction of bovine milk. By way of example, the whey-containing bovine milk fraction may comprise cheese whey. Generally, the exosomes are obtained from a whey-containing bovine milk fraction using gentle procedures which do not disrupt the exosome vesicle membrane, thereby leaving the exosomes intact and active bioactive agents contained within the exosome structure.

Various methods may be employed to isolate exosomes with care being exercised to avoid disruption of the lipid membrane. Fresh bovine milk, refrigerated bovine milk, thawed frozen bovine milk, or otherwise preserved bovine milk, or any bovine milk fraction containing exosomes, for example, cheese whey, may be employed as a source of exosomes. Isolating the exosomes may comprise performing the isolation immediately upon obtaining milk from a bovine. By way of example, isolating the exosomes may comprise performing the isolation within about 1 day, or about 2 days, or about 3 days, or about 4 days, or about 5 days or about 6 days, or about 7 days from the time of obtaining the milk from a bovine. The exosomes may be isolated within about 10 days, or within about 14 days from the time of obtaining milk from a bovine. Additionally, the bovine milk may be frozen and then thawed for processing for isolating exosomes, with the bovine milk preferably having been frozen within about 1 day, or about 2 days, or about 3 days, or about 4 days, or about 5 days or about 6 days, or about 7 days from the time of obtaining the milk from a bovine. Thawed milk is preferably processed immediately upon thawing. The fresh bovine milk may be subjected to the processing within about 5 days of obtaining the milk from a bovine, or thawed bovine milk which is subjected to processing is thawed from bovine milk that was frozen within about 5 days of obtaining the milk from a bovine.

As mentioned above, a whey-containing bovine milk fraction or, specifically, cheese whey, may serve as a source of exosomes. Cheese whey is the liquid by-product of milk after the formation of curd during the cheese-making or casein manufacturing process. Since cheese whey has already been separated from the casein fraction during the cheese manufacture process, cheese whey has very low casein content. Furthermore, cheese whey advantageously retains more than 50% of milk nutrients, including lactose, fat, proteins, mineral salts, and, surprisingly, a significant number of exosomes that were originally present in the milk in intact form. In addition to these benefits, cheese whey is less expensive than raw milk, and thus using cheese whey as a starting material significantly reduces costs for production of an exosome-enriched product. As such, cheese whey is a novel and promising source for isolating milk exosomes and producing exosome-enriched products.

In a specific embodiment, the cheese whey is obtained by applying an enzyme or enzyme mixture, and more specifically a protease enzyme, for example chymosin, to milk to hydrolyze casein peptide bonds, thus allowing for enzymatic coagulation of casein in the milk. Thus, when the protease enzyme cleaves the protein, it causes the casein in the milk to coagulate and form a gel structure. The casein protein gel network and milk fat then contract together and form curd. The resulting liquid that is separated from the curd is often referred to as sweet whey or cheese whey, typically has a pH from about 6.0 to about 6.5, and comprises whey proteins, lactose, minerals, water, fat and other low level components.

As indicated above, it is important that the enzyme or enzyme mixture is capable of destabilizing the casein protein in the milk fraction by cleaving peptides which stabilize the casein protein in the milk. Therefore, any proteolytic enzyme suitable for this purpose may be used to obtain cheese whey. In a preferred embodiment, however, the cheese whey is provided by adding rennet enzyme to bovine milk, resulting in enzymatic coagulation of casein. Rennet enzyme is commonly used in the cheese making process and comprises a set of enzymes which are produced in the stomachs of ruminant mammals. These enzymes normally include chymosin, pepsin, and lipase. The rennet enzyme mix destabilizes the casein protein in the bovine milk fraction by proteolytically cleaving peptides which stabilize the protein in the milk. As indicated above, the casein in the milk coagulates and contracts with milk fat to form the cheese curd. The remaining liquid, i.e., the sweet cheese whey, comprises whey proteins, lactose, minerals, water, fat, and other low level components.

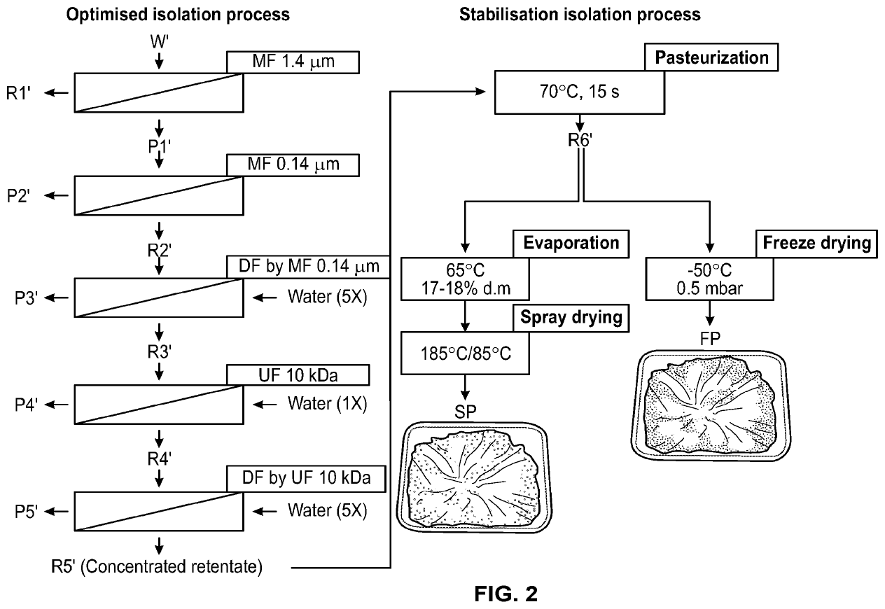

By way of example, a gentle procedure of obtaining an exosome-enriched product containing intact bovine milk-derived exosomes may comprise physical methods and/or chemical methods. In one embodiment, an exosome-enriched product is obtained by cascade membrane filtration. In a specific embodiment, the exosome-enriched product is lactose-free. In a specific embodiment, sweet cheese whey, which may be obtained as described in the preceding paragraph, is processed using tandem multiple ceramic filtration steps. In a specific embodiment, a multiple filtration process employs, successively, membranes with cut offs which gradually decrease in size. In a specific embodiment, the method of processing sweet cheese whey is subjected to microfiltration (MF, ultrafiltration (UF) and diafiltration (DF). In one more specific embodiment, as shown in FIG. 2, the process employs, successively, MF, UF and DF membranes with cut offs of about 1.4 μm, 0.14 μm and 10 kDa to provide an exosome-enriched product.

In a specific embodiment, the exosome-enriched product resulting from successive filtration steps may be pasteurized to provide storage stability. For example, the exosome-enriched product may be heated, for example, at about 70° C. for about 15 seconds, to ensure microbiological stability in order to yield a pasteurized fraction. Other pasteurization conditions will be apparent to those skilled in the art and may be employed.

With or without pasteurization, the exosome-enriched product may be used as is or subjected to additional processing steps to provide a desired physical form. In one embodiment, the exosome-enriched product, optionally pasteurized, may be converted to a powder form. In more specific embodiments, the exosome-enriched product can be spray-dried, freeze dried, or otherwise converted to powder form. In one specific embodiment, the exosome-enriched product may be spray dried, for example, at 185° C./85° C., to obtain an exosome-enriched product in the form of a spray-dried powder (SP). Prior to spray drying, the exosome-enriched product may be subjected to an optional evaporation step to increase the solids content of the product and therefore reduce the time and/or energy demand for the spray drying process. Other spray drying conditions will be apparent to those skilled in the art and may be employed. Alternatively, the exosome-enriched product may be freeze-dried, for example at −50° C. and 0.5 mbar vacuum to obtain an exosome-enriched freeze-dried powder (FP). Other freeze drying conditions will be apparent to those skilled in the art and may be employed.

In another specific embodiment, the exosome-enriched product comprises at least 0.001 wt % exosomes. In another specific embodiment, the exosome-enriched product comprises at least about 0.001 wt %, 0.01 wt %, 1 wt %, 5 wt %, 10 wt %, 15 wt %, 20 wt %, 25 wt %, 30 wt %, 35 wt %, 40 wt %, 45 wt %, or 50 wt % exosomes. In a further embodiment, the exosome-enriched product comprises at least about 10⁸ exosomes per gram of the exosome-enriched product as measured by a nanotracking procedure. Briefly, nanoparticle tracking analysis (NTA) can be used to determine exosome diameter and concentration. The principle of NTA is based on the characteristic movement of nanosized particles in solution according to the Brownian motion. The trajectory of the particles in a defined volume is recorded by a camera that is used to capture the scatter light upon illumination of the particles with a laser. The Stokes-Einstein equation is used to determine the size of each tracked particle. In addition to particle size, this technique also allows determination of particle concentration.

In a more specific embodiment, the exosome-enriched product comprises from about 10⁸ to about 10¹⁴ exosomes per gram of the exosome-enriched product. In yet a more specific embodiment, the exosome-enriched product comprises from about 10⁹ to about 10¹³ exosomes per gram of the exosome-enriched product. In another specific embodiment, the exosome-enriched product contains at least about a three-fold increase in the number of exosomes, as compared to a raw whey-containing bovine milk fraction. In a more specific embodiment, the exosome-enriched product contains a 3-fold to 50-fold increase in the number of exosomes, as compared to a raw whey-containing bovine milk fraction, for example cheese whey.

In another embodiment, at least about 50 wt % of the exosomes in the exosome-enriched product are intact. In a specific embodiment, at least about 55, 60, 65, 70, 75, 80, 85, 90, or 95 wt % of the exosomes in the exosome-enriched product are intact.

In one embodiment, the exosome-enriched product is administered in the form of an exosome-enriched powder. In another embodiment, the exosome-enriched product is administered in the form of an exosome-enriched liquid. The exosome-enriched product can be administered to the subject in either form.

In a specific embodiment, the exosome-enriched product comprising intact bovine milk-derived exosomes is administered to the subject at a dose of about 0.01 to about 30 g. More specifically, the dosage of the exosome-enriched product comprising the intact bovine milk-derived exosomes may be from about 0.1 to about 30 g, from about 0.1 to about 15 g, or from about 1 to about 15 g. The exosome-enriched product comprising the intact bovine milk-derived exosomes can be administered to a subject at any of the above dosages from about 1 to about 6 times per day or per week, or from about 1 to about 5 times per day or per week, or from about 1 to about 4 times per day or per week, or from about 1 to about 3 times per day or per week. By way of example, the dosage of the exosome-enriched product comprising the intact bovine milk-derived exosomes may be from about 0.01 to about 30 g/day, from about 0.1 to about 30 g/day, from about 0.1 to about 15 g/day, or from about 1 to about 15 g/day.

In a specific embodiment, the exosome-enriched product comprising the intact bovine milk-derived exosomes is administered to the subject orally.

In another specific embodiment, the subject is a human. In other embodiments, the subject is a human adult 20 years of age or older. By way of example, the subject may be an aging human adult, for example, over 25 years of age, over 30 years of age, over 35 years of age, over 40 years of age, over 45 years of age, over 50 years of age, over 55 years of age, over 60 years of age or older. In another embodiment, the subject is a human adult 65 years of age or older. By way of example, the subject may be an aging human adult, for example, over 70 years of age, over 75 years of age, over 80 years of age, over 85 years of age, or older. As indicated above, aging adults, and more particularly the elderly, exhibit diminished articular tissue integrity. As such, the methods of the invention are particularly suitable for adults over the age of 65 years old.

Given that joint injury and repetitive joint stress from overuse have been shown to contribute to early-onset osteoarthritis, the methods of the present invention are also suitable for administration to physically active individuals, particularly those participating in high impact exercise. Thus, in a specific embodiment, the exosome-enriched product is administered to the subject following exercise. In another specific embodiment, the exosome-enriched product is administered to the subject prior to exercise.

In another embodiment, the subject is suffering from a joint disease, joint injury after trauma, declining joint health as a result of normal aging, or a combination thereof. In a specific embodiment, the joint disease is selected from the group consisting of synovitis, spondyloarthritis, bursitis, infectious arthritis, rheumatoid arthritis, psoriatic arthritis, osteoarthritis, chondromalacia patellae, lupus, gout, juvenile idiopathic arthritis, and combinations of two or more thereof. In another specific embodiment, the subject is suffering from one or more symptoms selected from the group consisting of joint swelling, joint inflammation, joint pain, joint stiffness, joint degeneration, reduced mobility, and combinations of two or more thereof.

In one embodiment, the exosome-enriched product comprising intact bovine milk-derived exosomes is administered to the subject in a nutritional composition comprising protein, carbohydrate, and/or fat. In another embodiment, the nutritional composition comprises protein, carbohydrate, fat, and one or more nutrients selected from the group consisting of vitamins, minerals, and trace minerals.

Non-limiting examples of vitamins include vitamin A, vitamin B12, vitamin C, vitamin D, vitamin E, vitamin K, thiamine, riboflavin, pyridoxine, niacin, folic acid, pantothenic acid, biotin, choline, inositol, and/or salts and derivatives thereof, and combinations thereof. Non-limiting examples of minerals and trace minerals include calcium, phosphorus, magnesium, zinc, manganese, sodium, potassium, molybdenum, chromium, iron, copper, and/or chloride, and combinations thereof.

In another specific embodiment, the nutritional composition comprises about 0.001 to about 30 wt %, about 0.001 to about 10 wt %, about 0.001 to about 5 wt %, about 0.001 to about 1 wt %, about 0.01 to about 30 wt %, about 0.01 to about 10 wt %, about 0.01 to about 5 wt %, about 0.01 to about 1 wt %, about 0.1 to about 30 wt %, about 0.1 to about 10 wt %, about 0.1 to about 5 wt %, about 0.1 to about 1 wt %, about 1 to about 30 wt %, about 1 to about 10 wt %, or about 1 to about 5 wt % of the exosome-enriched product comprising the intact bovine milk-derived exosomes, based on the weight of the nutritional composition. In a specific embodiment, the nutritional composition comprises from about 0.001 to about 10 wt % of the intact bovine milk-derived exosomes, based on the weight of the nutritional composition.

In view of the exosome-enriched product also containing whey protein, the exosome-enriched product may be the sole source of protein in the nutritional composition. Nevertheless, additional protein sources can be included in the nutritional composition. In one embodiment, the protein comprises whole egg powder, egg yolk powder, egg white powder, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hemp proteins, flax seed proteins, earthworm proteins, insect proteins, one or more amino acids and/or metabolites thereof, or combinations of two or more thereof.

The one or a mixture of amino acids, which may be described as free amino acids, can be any amino acid known for use in nutritional products. The amino acids may be naturally occurring or synthetic amino acids. In a specific embodiment, the one or more amino acids and/or metabolites thereof comprise one or more branched chain amino acids or metabolites thereof. Examples of branched chain amino acids include arginine, glutamine leucine, isoleucine, and valine.

In another specific embodiment, the one or more branched chain amino acids or metabolites thereof comprise alpha-hydroxy-isocaproic acid (HICA, also known as leuic acid), keto isocaproate (KIC), β-hydroxy-β-methylbutyrate (HMB), and combinations of two or more thereof.

The nutritional composition may comprise protein in an amount from about 1 wt % to about 30 wt % of the nutritional composition. More specifically, the protein may be present in an amount from about 1 wt % to about 25 wt % of the nutritional composition, including about 1 wt % to about 20 wt %, about 2 wt % to about 20 wt %, about 1 wt % to about 15 wt %, about 1 wt % to about 10 wt %, about 5 wt % to about 10 wt %, about 10 wt % to about 25 wt %, or about 10 wt % to about 20 wt % of the nutritional composition. Even more specifically, the protein comprises from about 1 wt % to about 5 wt % of the nutritional composition, or from about 20 wt % to about 30 wt % of the nutritional composition.

In another embodiment, the carbohydrate comprises maltodextrin, hydrolyzed starch, modified starch, hydrolyzed cornstarch, modified cornstarch, polydextrose, dextrins, corn syrup, corn syrup solids, rice maltodextrin, brown rice mild powder, brown rice syrup, sucrose, glucose, fructose, lactose, high fructose corn syrup, honey, maltitol, erythritol, sorbitol, isomaltulose, sucromalt, pullulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, fiber, fruit puree, vegetable puree, isomalto-oligosaccharides, monosaccharides, disaccharides, tapioca-derived carbohydrates, inulin, and artificial sweeteners, or combinations of two or more thereof.

The nutritional composition may comprise carbohydrate in an amount from about 5 wt % to about 75 wt % of the nutritional composition. More specifically, the carbohydrate may be present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, about 40 wt % to about 70 wt %, or about 15 wt % to about 25 wt %, of the nutritional composition.

In another embodiment, the fat comprises algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, long chain polyunsaturated fatty acids, or combinations of two or more thereof.

The nutritional composition may comprise fat in an amount of from about 0.5 wt % to about 30 wt % of the nutritional composition. More specifically, the fat may be present in an amount from about 0.5 wt % to about 10 wt %, about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

The concentration and relative amounts of the sources of protein, carbohydrate, and fat in the exemplary nutritional compositions can vary considerably depending upon, for example, the specific dietary needs of the intended user. In a specific embodiment, the nutritional composition comprises a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition, and, more specifically, such composition is in liquid form. In another specific embodiment, the nutritional composition comprises a source of protein in an amount of about 10 wt % to about 25 wt %, a source of carbohydrate in an amount of about 40 wt % to about 70 wt %, and a source of fat in an amount of about 5 wt % to about 20 wt %, based on the weight of the nutritional composition, and, more specifically, such composition is in powder form.

In one embodiment, the nutritional composition is a liquid nutritional composition and comprises from about 1 to about 15 wt % of protein, from about 0.5 to about 10 wt % fat, and from about 5 to about 30 wt % carbohydrate, based on the weight of the nutritional composition.

In another embodiment, the nutritional composition is a powder nutritional composition and comprises from about 10 to about 30 wt % of protein, from about 5 to about 15 wt % fat, and from about 30 wt % to about 65 wt % carbohydrate, based on the weight of the nutritional composition.

In a specific embodiment, the nutritional composition comprises at least one protein comprising milk protein concentrate and/or soy protein isolate, at least one fat comprising canola oil, corn oil, coconut oil and/or marine oil, and at least one carbohydrate comprising maltodextrin, sucrose, and/or short-chain fructooligosaccharide.

The nutritional composition may also comprise one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

In specific embodiments, the nutritional composition has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1.

The nutritional composition may be formed using any techniques known in the art. In one embodiment, the nutritional composition may be formed by (a) preparing an aqueous solution comprising protein and carbohydrate; (b) preparing an oil blend comprising fat and oil-soluble components; and (c) mixing together the aqueous solution and the oil blend to form an emulsified liquid nutritional composition. The intact exosomes may be added at any time as desired in the process, for example, to the aqueous solution or to the emulsified blend. The intact exosomes may be dry blended in powder form with one or more dry ingredients, for example, for combined addition to a liquid composition or if a powdered nutritional product is desirable.

In a specific embodiment, the nutritional composition is administered in the form of a powder. In another specific embodiment, the nutritional composition is administered in the form of a liquid. The nutritional composition can be administered to the subject in either form.

When the nutritional composition is a powder, for example, a serving size is from about 40 g to about 60 g, such as 45 g, or 48.6 g, or 50 g, to be administered as a powder or to be reconstituted in from about 1 ml to about 500 ml of liquid.

When the nutritional composition is in the form of a liquid, for example, reconstituted from a powder or manufactured as a ready-to-drink product, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 225 ml, or about 237 ml, or about 500 ml.

In specific embodiments, the nutritional compositions comprising bovine milk-isolated exosomes are administered to a subject once or multiple times daily or weekly. In specific embodiments, the nutritional composition is administered to the subject from about 1 to about 6 times per day or per week, or from about 1 to about 5 times per day or per week, or from about 1 to about 4 times per day or per week, or from about 1 to about 3 times per day or per week. In specific embodiments, the nutritional composition is administered once or twice daily for a period of at least one week, at least two weeks, at least three weeks, or at least four weeks.

The following Examples demonstrate aspects of the inventive methods and are provided solely for the purpose of illustration. The Examples are not to be construed as limiting of the general inventive concepts, as many variations thereof are possible without departing from the spirit and scope of the general inventive concepts.

### EXAMPLES

### Example 1: Preparation and Characterization of Exosome-Enriched Products

This example describes a method of preparing an exosome-enriched product from cheese whey. The cheese whey was provided by adding rennet enzyme to bovine milk, resulting in enzymatic coagulation of casein and production of sweet cheese whey, as described above.

An exosome-enriched product containing about 10⁸ to 10¹⁴ intact bovine milk-derived exosomes per gram of the exosome-enriched product was prepared by cascade membrane filtration. First, 1,000 L of sweet cheese whey was processed using tandem multiple ceramic filtration steps. With reference to FIG. 2, the first microfiltration MF step employed a membrane with a molecular weight cut off of 1.4 μm, which yielded a first retentate R1 and a first permeate P1. The first permeate P1 was then subjected to an ultrafiltration step UF with a molecular weight cut off of 0.14 μm, which yielded a second retentate R2 and second permeate P2. About 5 volumes of water was added to one volume of the second retentate R2, and the diluted retentate was then passed through the 0.14 μm UF membrane again to remove at least part of the lactose and minerals. The resulting retentate R3 was then combined with an equal volume of water and diafiltered using a 10 kDa membrane to produce a fourth retentate R4. The retentate R4 was diluted with a volume of water five times that of the fourth retentate R4 and diafiltered a second time using the 10 kDa membrane to yield a concentrated retentate, R5. The lactose-free exosome-enriched product R5 was pasteurized at 70° C. for 15 seconds to ensure microbiological stability in order to yield a pasteurized exosome-enriched product R6. A portion of the pasteurized exosome-enriched product R6 was subjected to evaporation at about 65° C. to increase the solids content up to 17-18% and then spray-dried at 185° C./85° C. to obtain an exosome-enriched spray-dried product, SP. Another portion of the pasteurized exosome-enriched product R6 was freeze dried at −50° C. and 0.5 mbar to obtain an exosome-enriched freeze-dried product, FP.

The starting cheese whey, the second retentate R2, and the exosome-enriched products comprising intact bovine milk derived exosomes prepared as described above were analyzed to determine lactose and protein content, as set forth in Table 1 below.

TABLE 1
Lactose and protein composition of the exosome-enriched product.

| | Protein % (by | Protein % (by | | |
|---|---|---|---|---|
| Fractions | Milkoscan) | LECO) | Lactose % | Total Solids % |
| W | 1.39 ± 0.02 | 0.93 | 4.48 ± 0.01 | 6.33 ± 0.03 |
| R2 | 1.82 ± 0.01 | 1.13 | 3.41 ± 0.02 | 5.62 ± 0.01 |
| R6 | 5.63 ± 0.04 | 6.87 | 0 | 7.10 ± 0.03 |
| SP | — | 80.34 | 0 | Powder |
| FP | — | 78.45 | 0 | Powder |

Composition analysis of different fractions and exosome-enriched powders:
W = cheese whey.
R2 = final exosome-enriched liquid fraction.
R6 = final exosome-enriched liquid fraction.
SP = spray-dried powder.
FP = freeze-dried powder.

The amount of fat, protein, lactose, and total solids of the collected samples from each of the fractions referred to in Table 1 were determined by Fourier transform infrared (FTIR) spectroscopy using a Bentley Instruments Dairy Spec FT (Bentley Instruments, Inc., Chaska, MN, USA). The Bentley Instruments Dairy Spec FT captures the complete infrared absorption spectrum of the milk sample for component analysis. This particular technology exceeds the IDF 141C:2000 Standard and ICAR requirements for Milk Component Measurement and uses AOAC approved methodology, thus providing a non-destructive, reliable and precise measurement.

The results presented in Table 1 surprisingly demonstrate that the pasteurized exosome-enriched product R6, the spray-dried exosome-enriched product SP, and the freeze-dried exosome-enriched product FP were all lactose-free. Further, the protein content in the pasteurized exosome-enriched product R6 increased almost 7 times with respect to the cheese whey starting material, and about 6 times with respect to the exosome-enriched second retentate R2. In addition, about 80% of the dry matter of the powders was protein and about 15% of the dry matter was fat, which is consistent with the lipid-protein nature of exosomes.

In order to gain further insight on the exosome content of the pasteurized exosome-enriched product R6 and the exosome-enriched SP and FP powders, a Western blot analysis was performed to detect the presence of the exosome-specific marker TSG101. The exosome-enriched product R6 and the exosome-enriched SP and FP powders showed the TSG101 band of interest at around 50 kDa. Notably, the TSG101 biomarker was not detectable in cheese whey, despite equal amounts of protein being loaded per lane. This indicates that the pasteurized exosome-enriched product R6 and the exosome-enriched SP and FP powders produced according to the process described above are significantly enriched in milk exosomes.

Transmission electron microscopy (TEM) was also used for purposes of assessing the presence of exosomes in the pasteurized exosome-enriched product R6, and in the exosome-enriched SP and FP powders. TEM is a technique which can be used for the direct visualization of nanosized structures, such as exosomes. The application of uranyl acetate as a negative dye was used to study the impact of thermal treatments, such as pasteurization, evaporation, spray-drying, and freeze-drying, on the exosome structure of the exosomes in the pasteurized lactose-free exosome-enriched product R6, and in the final lactose-free exosome-enriched SP and FP products. Briefly, the uranyl acetate acts as a negative dye, which stains the background and leaves the intact vesicular structures, such as intact exosomes, unstained and highly visible.

The lactose-free exosome-enriched SP and FP powders prepared as described above were resuspended in water and 3 microliters of each sample were placed on a Formvar® coated grid and stained with 2% uranyl acetate for 5 minutes. The exosome-enriched R5 and R6 products, prepared as described above, were placed undiluted on a Formvar® coated grid and stained with 2% uranyl acetate for 5 minutes. The samples were visualized at a magnification of ×25,000. TEM images of the R5 and R6 exosome-enriched products, and the exosome-enriched SP and FP powders showed that the intact exosomes were present at high concentration. Remarkably, none of the thermal treatments that were applied led to significant exosome damage. These results demonstrate that the process described above can isolate and stabilize a significant amount intact milk exosomes from cheese whey.

The exosome-enriched products comprising intact bovine milk-derived exosomes prepared as described above were also analyzed to determine nucleic acid content. More specifically, the exosome-enriched SP and FP powders and the pasteurized exosome-enriched product R6 were analyzed in order to determine their total RNA content (μg), total miRNA content (μg), and miRNA as a percentage of the total RNA, as set forth in Table 2 below. 10 mg of each sample were extracted and analyzed using a Bioanalyzer 2100/Eukaryote Total RNA Nano Chip. The exosome-enriched SP and FP powders and the pasteurized exosome-enriched product R6 displayed high amounts of both RNA and miRNA, however the exosome-enriched SP powder showed higher miRNA content than the exosome-enriched FP powder. This indicates that spray-drying may be a better stabilization strategy for providing an exosome-enriched product in powder form.

TABLE 2
Nucleic acid composition of the exosome-enriched product.

| | Total RNA content | miRNA % (of total | miRNA content |
|---|---|---|---|
| | (μg) | nucleic acids) | (μg) |
| R6 | 5.50 | 67.1 | 3.68 |
| SP | 5.09 | 72.5 | 3.69 |
| FP | 2.51 | 76.1 | 1.91 |

The exosome-enriched products comprising intact bovine milk-derived exosomes were also analyzed to determine lipid composition. Ultra-performance liquid chromatography coupled to time-of-flight mass spectrometry analysis (UPLC-TOF-MS) was performed to analyze the lipid content of the lactose-free exosome-enriched products described above. The results are set forth in Table 3 below and are expressed as a percentage of total lipids.

TABLE 3
Lipid composition of the lactose-free exosome-enriched product.

| | R6 | | SP | | FP | |
|---|---|---|---|---|---|---|
| | % of total | mg/g | % of total | mg/g | % of total | mg/g |
| LIPID SPECIE | lipids | powder | lipids | powder | lipids | powder |
| Triacylglicerols | 76.9 | NA (liquid) | 70.6 | 117.4 | 70.7 | 75.1 |
| Phosphatidylcholine | 6.4 | NA (liquid) | 7.5 | 12.5 | 7.9 | 8.3 |
| Phosphatidylethanolamine | 5.0 | NA (liquid) | 6.7 | 11.1 | 6.0 | 6.3 |
| Sphingomyelin | 3.8 | NA (liquid) | 4.9 | 8.2 | 6.6 | 7.0 |
| Gangliosides (GD3) | 1.9 | NA (liquid) | 2.7 | 4.4 | 2.4 | 2.5 |
| Phosphatidylserine | 1.8 | NA (liquid) | 2.0 | 3.4 | 2.0 | 2.1 |
| Cholesterol esters | 1.2 | NA (liquid) | 1.4 | 2.4 | 1.1 | 1.2 |
| Ceramide dihexoside | 0.9 | NA (liquid) | 1.3 | 2.2 | 1.0 | 1.1 |
| Dihydrosphingomyelin | 0.6 | NA (liquid) | 0.8 | 1.3 | 0.8 | 0.8 |
| Cholesterol (free) | 0.4 | NA (liquid) | 0.5 | 0.8 | 0.4 | 0.4 |
| Ceramide monohexoside | 0.3 | NA (liquid) | 0.4 | 0.7 | 0.4 | 0.4 |
| Ether-linked | 0.2 | NA (liquid) | 0.2 | 0.4 | 0.2 | 0.2 |
| phosphatidylethanolamine | | | | | | |
| Ceramide | 0.1 | NA (liquid) | 0.2 | 0.3 | 0.2 | 0.2 |
| Gangliosides (GM3) | 0.1 | NA (liquid) | 0.2 | 0.3 | 0.1 | 0.1 |
| Phosphatidylinositol | 0.1 | NA (liquid) | 0.2 | 0.3 | 0.1 | 0.1 |
| Lysophosphatidylethanolamine | 0.1 | NA (liquid) | 0.1 | 0.2 | 0.1 | 0.1 |
| Lysophosphatidylcholine | 0.1 | NA (liquid) | 0.1 | 0.2 | 0.1 | 0.1 |
| Dihydroceramide | 0.1 | NA (liquid) | 0.1 | 0.1 | 0.1 | 0.1 |
| Free fatty acids | Traces | Traces | Traces | Traces | Traces | Traces |

| | | | |
|---|---|---|---|
| Total lipid content | NA | NA | NA |
| (1%, on a dry basis) | | | |

The protein compositions of the exosome-enriched products were also determined. Specifically, the protein composition was determined by LC-MS/MS and mass spectra were searched in Proteome Discoverer v1.4 (database Bos Taurus, Uniprot 06/19+Proteomics contaminants database). The results of several proteins of interest are set forth in Table 4 and surprisingly demonstrate that caseins were present at very low levels (e.g., only 0.04% of a α-S2-casein was detected). In addition, the results demonstrate that significant amounts of bioactive proteins (i.e., lactoferrin and immunoglobulins) were detected. The results are expressed as % of total proteins identified.

TABLE 4
Protein composition of the lactose-free exosome-enriched product.

| | R6 | | SP | | FP | |
|---|---|---|---|---|---|---|
| | % of total | mg/g | % of total | mg/g | % of total | mg/g |
| PROTEIN | proteins | powder | proteins | powder | proteins | powder |
| β-lactoglobulin | 56.40 | NA (liquid) | 58.97 | 471.76 | 59.24 | 473.92 |
| Serum albumin | 12.12 | NA (liquid) | 11.77 | 94.16 | 12.32 | 98.56 |
| Antibodies (IgG, IgM, IgA) | 6.33 | NA (liquid) | 6.63 | 53.04 | 6.42 | 51.36 |
| α-lactalbumin | 5.00 | NA (liquid) | 4.55 | 36.4 | 4.59 | 36.72 |
| Lactoferrin | 3.53 | NA (liquid) | 3.12 | 24.96 | 3.18 | 25.44 |
| Butyrophilin | 2.18 | NA (liquid) | 1.90 | 15.2 | 1.06 | 8.48 |
| Lactadherin | 1.67 | NA (liquid) | 1.45 | 11.6 | 1.52 | 12.16 |
| Xanthine | 1.45 | NA (liquid) | 1.36 | 10.88 | 1.40 | 11.2 |
| dehydro-genase/oxidase | | | | | | |
| Transferrin | 1.27 | NA (liquid) | 1.05 | 8.4 | 0.98 | 7.84 |
| Lactoperoxidase | 0.54 | NA (liquid) | 0.53 | 4.24 | 0.59 | 4.72 |
| Vitamin D-binding protein | 0.17 | NA (liquid) | 0.15 | 1.2 | 0.15 | 1.2 |
| Osteopontin | 0.18 | NA (liquid) | 0.15 | 1.2 | 0.15 | 1.2 |
| α-S1-casein | 0.29 | NA (liquid) | 0.13 | 1.04 | 0.21 | 1.68 |
| κ-casein | 0.06 | NA (liquid) | 0.04 | 0.32 | 0.05 | 0.4 |
| β-casein | 0.04 | NA (liquid) | 0.03 | 0.24 | 0.03 | 0.24 |
| α-S2-casein | 0.02 | NA (liquid) | 0.01 | 0.08 | 0.01 | 0.08 |
| Total casein | 0.41 | NA (liquid) | 0.21 | 1.68 | 0.30 | 2.4 |

| | | | |
|---|---|---|---|
| Total protein content | NA | NA | NA |
| (1%, on a dry basis) | | | |

### Example 2: Increased Proteoglycan Synthesis in C28/12 Human Chondrocyte Cells Incubated with Intact Bovine Milk-Derived Exosomes

This example demonstrates that an exosome-enriched product containing intact bovine milk-derived exosomes increases proteoglycan synthesis in human chondrocyte cells. Proteoglycan production was analyzed by the Alcian blue staining method. The principle of the assay is based on the strong interaction between the tetravalent cationic dye Alcian blue and the negatively charged glycosaminoglycans, which decorate proteoglycan core proteins.

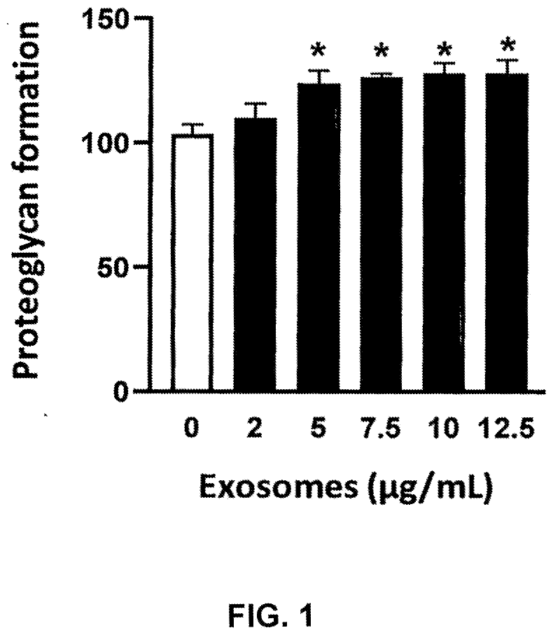

C28/12 human chondrocyte cells were grown in Dulbecco's modified Eagle's medium (DMEM), containing 10% fetal bovine serum and antibiotics (50 U/mL penicillin and 50 μg/mL streptomycin) in 5% CO₂ at 37° C. The cells were subcultured after reaching 70-90% confluence. The spray dried (SP) exosome-enriched product comprising the intact bovine milk-derived exosomes, which was obtained by the procedure set forth in the preceding paragraph, was resuspended in PBS. The cells were treated with increasing amounts of the SP exosome-enriched product comprising intact bovine milk-derived exosomes for 48 hours, as set forth in FIG. 1.

As mentioned above, proteoglycan production was analyzed by Alcian blue staining. The cells were incubated with or without the exosome-enriched product comprising intact bovine milk-derived exosomes in growth medium for 48 hours. The cells were then washed with PBS and fixed with 2% paraformaldehyde prior to staining with 1% Alcian blue solution in 3% acetic acid for 30 minutes. The cells were then washed with water and air dried. Colorant was extracted from the cells by adding 10% acetic acid solution. The absorbance of the colored solution was measured at 640 nm.

As shown in FIG. 1, compared to untreated C28/12 human chondrocyte cells, the C28/12 human chondrocyte cells incubated in bovine milk-derived exosomes exhibited increased proteoglycan synthesis at 48 hours, with statistically significant increases in proteoglycan synthesis observed when the cells were incubated with 5, 7.5, 10 and 12.5 μg/mL of the exosome-enriched product comprising intact bovine milk-derived exosomes. The highest increase in proteoglycan synthesis, as compared to control, was observed at 10 and 12.5 μg/mL of the exosome-enriched product comprising intact bovine milk-derived exosomes. The observed increase in proteoglycan synthesis indicates that the exosome-enriched product comprising intact bovine milk-derived exosomes is a novel tool to increase chondrocyte proteoglycan synthesis, and useful to enhance cartilage repair and recovery.

These results thus indicate that the exosome-enriched product comprising intact bovine milk-derived exosomes can enhance cartilage repair and recovery, which is particularly relevant for the treatment of conditions such as joint disease, joint injury after trauma, declining joint health as a result of normal aging, or a combination thereof.

While the present invention has been illustrated by the description of embodiments thereof, and while the embodiments have been described in considerable detail, such descriptions are not intended to restrict or in any way limit the scope of the appended claims to such detail. Additional advantages and modifications will readily appear to those skilled in the art. Therefore, the invention, in its broader aspects, is not limited to the specific details, the representative compositions and processes, or illustrative examples shown and described. Accordingly, departures may be made from such details without departing from the spirit or scope of the general inventive concept.

1. A method of improving joint health in a subject in need thereof, comprising administering an exosome-enriched product comprising intact bovine milk-derived exosomes to the subject in need thereof.

2. (canceled)

3. The method of claim 1, wherein the exosome-enriched product comprises at least 0.001 wt % exosomes.

4. (canceled)

5. (canceled)

6. The method of claim 1, wherein at least about 50, 55, 60, 65, 70, 75, 80, 85, 90, or 95 wt % of the exosomes in the exosome-enriched product are intact.

7. The method of claim 1, wherein the exosome-enriched product comprises at least 0.001 wt % exosomes, wherein at least about 50 wt % of exosomes in the exosome-enriched product are intact, and/or wherein the exosome-enriched product is lactose-free.

8. (canceled)

9. (canceled)

10. The method of claim 1, wherein the exosome-enriched product comprising the intact bovine milk-derived exosomes is administered to the subject at a dose of about 0.01 to about 30 g.

11. (canceled)

12. (canceled)

13. The method of claim 1, wherein the subject is 65 years of age or older.

14. (canceled)

15. (canceled)

16. The method of claim 1, wherein the subject is suffering from a joint disease, joint injury after trauma, declining joint health as a result of normal aging, articular cartilage lesions, or a combination thereof.

17. The method of claim 16, wherein the joint disease is selected from the group consisting of synovitis, spondyloarthritis, bursitis, infectious arthritis, rheumatoid arthritis, psoriatic arthritis, osteoarthritis, chondromalacia patellae, lupus, gout, juvenile idiopathic arthritis, and combinations of two or more thereof.

18. The method of claim 1, wherein the subject is suffering from one or more symptoms selected from the group consisting of joint swelling, joint inflammation, joint pain, joint stiffness, joint degeneration, reduced mobility, and combinations of two or more thereof.

19. The method of claim 1, wherein the exosome-enriched product comprising intact bovine milk-derived exosomes is administered to the subject in a nutritional composition comprising protein, carbohydrate, and/or fat.

20. The method of claim 19, wherein the nutritional composition comprises protein, carbohydrate, fat, and one or more nutrients selected from the group consisting of vitamins and minerals.

21. The method of claim 19, wherein the nutritional composition comprises from about 0.001 to about 30 wt % of the exosome-enriched product comprising the intact bovine milk-derived exosomes, based on the weight of the nutritional composition.

22. The method of claim 19, wherein the protein comprises whole egg powder, egg yolk powder, egg white powder, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hemp proteins, flax seed proteins, earthworm proteins, insect proteins, one or more amino acids and/or metabolites thereof, or combinations of two or more thereof.

23. The method of claim 22, wherein the one or more amino acids and/or metabolites thereof comprise one or more branched chain amino acids or metabolites thereof.

24. The method of claim 23, wherein the one or more branched chain amino acids or metabolites thereof comprise alpha-hydroxy-isocaproic acid (HICA), keto isocaproate (KIC), β-hydroxy-β-methylbutyrate (HMB), and combinations of two or more thereof.

25. The method of claim 19, wherein the nutritional composition comprises from about 1 wt % to about 30 wt %, from about 1 wt % to about 25 wt %, from about 1 to about 20 wt %, from about 1 to about 15 wt %, from about 1 to about 10 wt %, or from about 10 wt % to about 30 wt % protein, based on the weight of the nutritional composition.

26. The method of claim 19, wherein the carbohydrate comprises maltodextrin, hydrolyzed starch, modified starch, hydrolyzed cornstarch, modified cornstarch, polydextrose, dextrins, corn syrup, corn syrup solids, rice maltodextrin, brown rice mild powder, brown rice syrup, sucrose, glucose, fructose, lactose, high fructose corn syrup, honey, maltitol, erythritol, sorbitol, isomaltulose, sucromalt, pullulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, fiber, fruit puree, vegetable puree, isomalto-oligosaccharides, monosaccharides, disaccharides, tapioca-derived carbohydrates, inulin, and artificial sweeteners, or combinations of two or more thereof.

27. The method of claim 19, wherein the nutritional composition comprises from about 5 wt % to about 75 wt %, from about 5 wt % to about 70 wt %, from about 5 wt % to about 65 wt %, from about 5 wt % to about 50 wt %, from about 5 wt % to about 40 wt %, from about 5 wt % to about 30 wt %, from about 5 wt % to about 25 wt %, from about 10 wt % to about 65 wt %, from about 20 wt % to about 65 wt %, from about 30 wt % to about 65 wt %, from about 40 wt % to about 65 wt %, or from about 15 wt % to about 25 wt % carbohydrate, based on the weight of the nutritional composition.

28. The method of claim 19, wherein the fat comprises algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, long chain polyunsaturated fatty acids, or combinations of two or more thereof.

29. The method of claim 19, wherein the nutritional composition comprises from 0.5 wt % to 20 wt %, from about 0.5 to about 15 wt %, from about 0.5 to about 10 wt %, from about 0.5 to about 5 wt %, or from about 5 to about 15 wt % fat, based on the weight of the nutritional composition.

30. (canceled)

31. (canceled)

32. The method of claim 19, wherein the nutritional composition comprises from about 1 to about 15 wt % of protein, from about 0.5 to about 10 wt % fat, and from about 5 to about 30 wt % carbohydrate, based on the weight of the nutritional composition, wherein the nutritional composition is in the form of a liquid.

33. The method of claim 19, wherein the nutritional composition comprises from about 10 to about 30 wt % of protein, from about 5 to about 15 wt % fat, and from about 30 wt % to about 65 wt % carbohydrate, based on the weight of the nutritional composition, wherein the nutritional composition is in the form of a powder.

34. The method of claim 19, wherein the nutritional composition comprises at least one protein comprising milk protein concentrate and/or soy protein isolate, at least one fat comprising canola oil, corn oil, coconut oil and/or marine oil, and at least one carbohydrate comprising maltodextrin, sucrose, and/or short-chain fructooligosaccharide.
